(19) United States
(12) Patent Application Publication
(10) Pub. No.: US 2013/0023971 A1
(43) Pub. Date: Jan. 24, 2013

(54) Flexible Evaporator Coil For Application of Cold Directly to Unevenly Shaped Objects (formerly Refrigerated Head Gear for Super Cold Therapy)

(71) Applicant: Thomas Benjamin Smiley, Carlsbad, CA (US)

(72) Inventor: Thomas Benjamin Smiley, Carlsbad, CA (US)

(21) Appl. No.: 13/184,818

(22) Filed: Jul. 18, 2011

(51) Int. Cl. F28F 1/00; A61F 7/10; F25B 1/00

(52) U.S. Cl. 607/114; 165/177; 62/498

(57) ABSTRACT

A flexible and pliable evaporator coil embedded in freezer gel and surrounded by a flexible enclosure. In its warm state, the evaporator coil/gel enclosure can be fitted around a variety of shapes such as legs, arms, heads, or devices requiring cold or super cold in direct surface contact. The evaporator coil is designed to be connected directly to a traditional refrigeration system employing refrigerant, a compressor, and condenser coil.

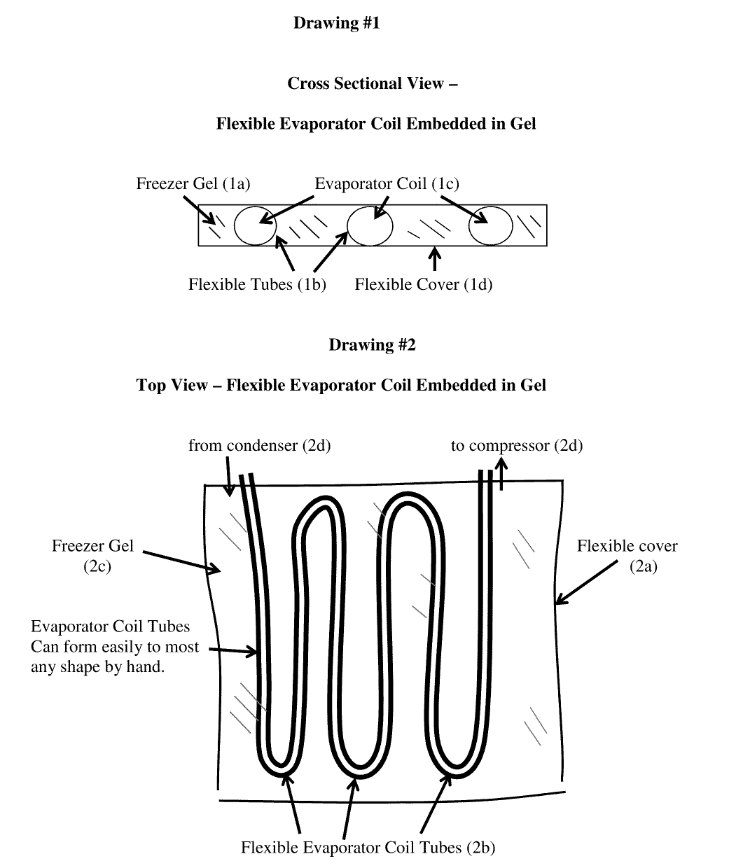

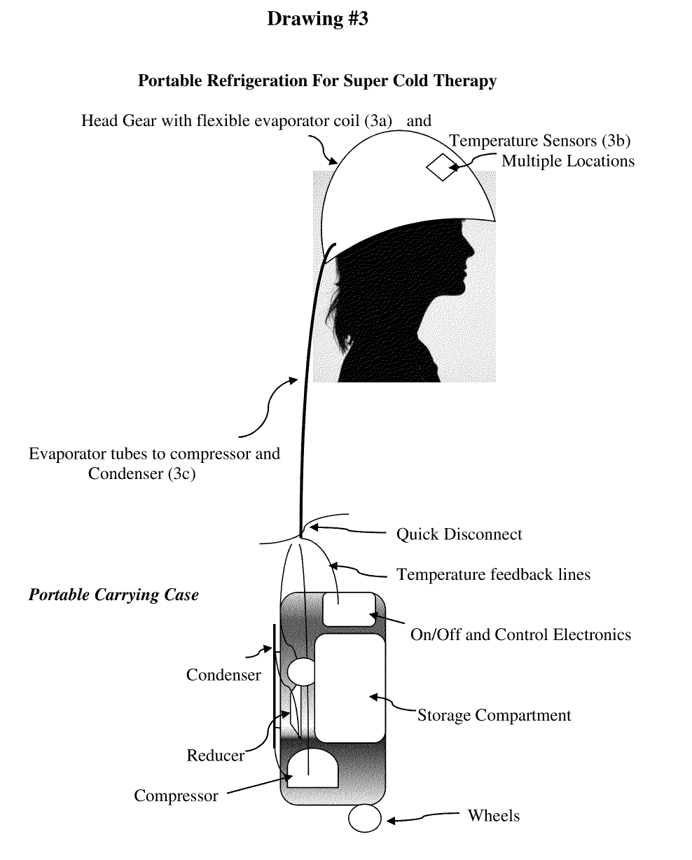

### CROSS REFERENCE

McKay #U.S. Pat. No. 5,305,470

Tremblay #U.S. Pat. No. 5,469,579

Fronda #U.S. Pat. No. 5,897,581

Truelock #U.S. Pat. No. 4,382,446 Leong, et.al. #U.S. Pat. No. 5,950,234

Lee, Carole NUS2002/0058976 A1

Culp #U.S. Pat. No. 2,158,571

### Inventors: Thomas B. Smiley, Christine L. Smiley Carlsbad, Calif.

### BACKGROUND OF THE INVENTION

The application of cold directly to an object or to the human body for purposes of healing or convenience is well known and in wide practice. In all cases this is done either with the circulation of chilled water, circulation of chilled air, or direct application of freezer gels which have been pre-chilled and then placed directly on the body part or object to be chilled.

This has proven to be an imperfect process in many ways. Circulation of chilled air cannot provide temperatures low enough to accomplish the objectives in many cases, chilled water similarly cannot transfer enough heat out to be effective enough for many purposes, and freezer gels must be changed often to maintain the desired cold temperature of the objective. This invention significantly improves upon all of these currently existing methods.

### SUMMARY OF THE INVENTION

The invention disclosed solves these problems and provides a convenient system for applying the cold directly, and with precision, to any shaped body part or any shaped object.

The invention, in its preferred embodiment, provides for a flexible and easily shapeable evaporator coil (Drawing #1) embedded inside a flexible gel filled container and connected to a portable refrigeration system. This flexible evaporator coil can be shaped to any surface thereby applying cold or, heat when operated in reverse direction, directly to almost any curved surface of any configuration. It is flexible and pliable such that it can be easily bent by hand hundreds of times over its natural life. Until this invention, evaporator coils in refrigeration systems have been constructed from rigid tubes. These rigid evaporator tubes remove heat (transfer cold) from carrier mediums such as air or liquids at some distance removed from the actual object desired to be chilled. The carrier mediums deliver the cold to the desired object. The carrier mediums used are water, air, gel solutions, or other material capable of being transported over some distance. A rigid evaporator coil is prevented from making direct contact with objects of many different shapes and sizes, hence necessitating this carrier medium. The carrier medium provides the final point of contact to the object intended to be chilled.

The invention described, a flexible and pliable evaporator coil, can conform to most any shape and delivers cold directly to the desired object eliminating the need for the carrier medium. The preferred embodiment of the flexible evaporator coil (1a) is constructed of a flexible hose or tube (1b) made from any material capable of bending in any direction while in a room temperature condition, such material shall withstand the pressure of refrigerant without fear of leaking, it can withstand multiple flexures without leaking, and it is capable of transferring temperature. The hose or tube is either embedded in freezer gel (1c), or adjacent to freezer gel and enclosed in a flexible pouch (1d) or container of any appropriate configuration. The container is designed to be in direct contact with the surface to be cooled. The gel provides a medium for spreading the cold, and in its warm state, can be fitted to most any odd shape. This makes it ideal for use as a method to apply direct super cold to uneven surfaces. As the evaporator coil removes heat from the gel, the gel stiffens and maintains its now custom formed shape. This is particularly important in many desired applications where very consistent and thorough application of cold is required. Several specific applications which will benefit from this invention are described below:

Specific Application #1—Chemotherapy induced Hair Loss Prevention (Drawing #2).

In an effort to apply very cold therapy to the skull for the purpose of reducing hair loss during chemotherapy, pre-frozen gel filled caps have been employed with some success.

The reason this is successful is that chemotherapy drugs kill fast growth cells and, hair follicles which are fast growth cells, are then almost immediately lost. Chilling the skull down to −20 degrees C. (−4 deg F) during chemotherapy, and for a period after treatment, reduces blood flow to the skull thereby reducing or eliminating absorption of the drugs into those hair follicles.

Currently employed solutions for eliminating hair loss during chemotherapy employed pre-frozen gel filled caps. These caps are designed to be placed on the head before, during, and after the chemotherapy session. The problem with the pre-frozen cap design is in fitting the very cold, very stiff gel filled cap evenly, and maintaining the correct temperature over the required period of time.

All of these caps require extreme effort and attention to details over the 6 hours necessary to accomplish their objective. All such caps require a separate freezer unit to freeze 15 to 20 different caps all at beginning temperatures of −31 deg C. In use, they must be changed every 30 minutes and replaced with a freshly chilled cold cap from the freezer to maintain the necessary cold temperature window on the scalp. Each time a new cap is applied, the scalp has the opportunity to warm slightly which can allow blood flow back to the hair follicles and then hair loss. Each newly applied cap must be at the exact temperature. Too low will freezer burn the scalp, too high will allow blood flow. For this treatment to work, every surface of the scalp must be covered. All head shapes are different and therefore extra time and effort is required to correctly position the cap on the patient. Removing and replacing 15 caps quickly and correctly is very difficult and prone to error. The patient needs a dedicated assistant to change caps. The described invention solves all of these problems.

Specific Application #2—Muscle therapy.

Similarly to hair loss prevention, applying cold directly to an arm or leg, or other body part is very effective in promoting healing and pain reduction. Professional athletes, as well as weekend athletes, when experiencing muscle injury, must wrap themselves in ice packs or sit with an ice chest which is connected to a pump that moves ice (carrier medium) across the limb. These are cumbersome, inexact in the amount of cold they deliver, and require replenishment. Our invention solves this problem and has the added advantage of changing from hot to cold by reversing the direction of the refrigerant so that the flexible (evaporator) coil becomes the condenser and the rigid (condenser) becomes the cold evaporator coil.

### Specific Application #3—Beverage Cooling

Kegs of beer are best maintained within a narrow range of temperature to assure freshness and quality. In the case of tailgate parties they can either be wrapped in insulating blankets, placed in an appropriately sized refrigerator which will operate from 12v DC provided by a vehicle, or placed in a tub of ice. These methods are cumbersome for the user, and inexact in their ability to control the temperature. The described flexible evaporator coil invention, when appropriately sized and wrapped around a keg of beer, more efficiently keeps the keg cold, is more portable, and lighter than a fully enclosed small refrigerator, and further will consume less space.

### DESCRIPTION OF DRAWINGS

Drawing #1 is a cross sectional view of the flexible evaporator coil inside the flexible container surrounded by freezer gel.
1a shows the freezer gel throughout the interior of the encasement.
1b shows the flexible tubes carrying refrigerant from the condenser or compressor depending upon the direction of flow.
1c shows evaporator coil tubes embedded in the gel.
1d shows the flexible container holding the coil and gel.

Drawing #2 is a top view of the flexible container with the flexible evaporator tubes inside in a distribution pattern that evenly spreads the cold (or heat) throughout the container.
2a shows the container
2b shows the evaporator tubes
2c shows the freezer gel throughout the container and surrounding the tubes
2d and 2e show the tubes exiting the container so that they can connect to both the compressor and the condenser coil.

Drawing #3 is a side view of an embodiment of the invention in use to prevent hair loss during chemotherapy.
3a shows the flexible evaporator coil inside a container cap fitted on a patient's head.
3b shows temperature sensors inside the cap which can provide precise temperature feedback in order to maintain the correct temperature range.
3c. shows the connection of the evaporator coil tubes to the portable refrigeration unit.

### DETAILED DESCRIPTION OF THE INVENTION

Refrigeration systems are well established as cost effective means to cool and freeze material with consistency and accuracy. Portable systems are currently available which are light in weight and can be operated from both AC and DC voltages. Refrigeration systems can also be operated in reverse direction to provide heat to the evaporator coil.

Our invention utilizes traditional compressor-condenser-reducer valve-evaporator technology with the exception that the evaporator coil (2a) is constructed from flexible and easily pliable hose (2c) capable of withstanding the pressure necessary from the compressor, and constructed from materials which not disintegrate with exposure to the common refrigerants. In the preferred embodiment, the evaporator coil is embedded in a gel and surrounded by a flexible container. Alternative embodiments have the flexible evaporator coil adjacent to the gel or other material suitable for spreading the cold or heat. The flexible container with evaporator coil inside when at room temperature or above room temperature can be adjusted by hand to conform firmly to most any shape. This pliability allows direct contact on all of the uneven surfaces of the intended object or human body part and minimizes the potential for small areas to be left untreated and maximizes the absorption of heat. This is a critical feature in the case of preventing hair loss during chemotherapy as every hair follicle allowed to rise in temperature will draw in the toxic drug and die giving up the hair it has produced. The major drawback of existing freezer gel caps is that they must go on the head in a frozen state and are very difficult to form into all of the unique curves of the human head.

In contrast, the described invention becomes firm in the correct shape as the evaporator coil extracts heat from the gel, and the cold spreads evenly throughout the evaporator coil container. The gel provides the consistency of temperature as the freezer unit cycles on and off. This effect promotes constancy of the temperature.

Temperature sensors can be attached to the interior of the container and pressed against the surface to be cooled. This technique provides an automatic feedback mechanism for cycling the freezer unit on and off and maintaining a closely monitored temperature on the desired surface.

While the impetus for the invention is super cold cap therapy for chemotherapy patients, a flexible and pliable evaporator coil embedded in freezer gel will be useful in any application not served by fixed evaporator coils, and not served by cold air flow. The improved technology will also be useful in managing injuries which require alternating heat and cold by utilizing the common technique of reversing coolant flow direction and creating a heat source of the evaporator coil. The flexible evaporator coil disclosed is most effective in applications when space is limited, where thorough direct contact is desired, or where subzero temperatures must be maintained.

1. A flexible evaporator coil embedded in or conjoined with a gel solution which is pliable down to −20 degrees C. and capable of multiple cycles of heat and cold.

2. A flexible evaporator coil as in above which is further encased in a flexible container capable of conforming to a variety of surfaces with curves and angles.

3. A refrigeration system employing a compressor, refrigerant, a condenser, and an evaporator. Of which the evaporator section is imbedded in freezer gel and surrounded by an enclosure to prevent the gel from flowing away from the evaporator.

4. A flexible enclosure held in direct contact with the skin to apply super cold therapy to the body, whereby in its warm state it can formed to any body part, and in its frozen state can apply even, consistent, super cold to the skin surface.

5. Use of automated refrigeration technology to improve the application of cold therapy for chemotherapy patients who desire to reduce or eliminate the hair loss associated with cancer drugs.

6. A method of applying automated refrigeration technology to improve the efficiency and portability of delivering cold therapy directly to human tissue.

7. A method for applying a flexible and portable cold surface directly to any object.

8. Application of an evaporator coil connected to an automated refrigeration unit and wrapped around a beer keg or other products/objects to provide consistent and constant refrigeration while physically away from standard coolers.
